(19) United States
(12) Patent Application Publication
(10) Pub. No.: US 2025/0197600 A1
(43) Pub. Date: Jun. 19, 2025

(54) CITRIC ACID-GLYCIDYL ETHER-BUTYL ESTER PLASTICIZER AND PREPARATION METHOD THEREOF

(71) Applicant: BEIJING TECHNOLOGY AND BUSINESS UNIVERSITY, Beijing (CN)

(72) Inventors: Yunxuan WENG, Beijing (CN); Caili ZHANG, Beijing (CN); Xiaoqian DIAO, Beijing (CN); Shiyan SUN, Beijing (CN); Yu HAN, Beijing (CN)

(73) Assignee: BEIJING TECHNOLOGY AND BUSINESS UNIVERSITY, (omitted)

(21) Appl. No.: 18/921,433

(22) Filed: Oct. 21, 2024

(30) Foreign Application Priority Data
Dec. 18, 2023 (CN) 202311736271.9

(51) Int. Cl. C08K 5/1515; C07D 301/02; C07D 303/30

(52) U.S. Cl. CPC C08K 5/1515; C07D 301/02; C07D 303/30

(57) ABSTRACT

A citric acid-glycidyl ether-butyl ester plasticizer and a preparation method thereof are provided. The citric acid-glycidyl ether-butyl ester plasticizer includes citric acid-diglycidyl ether-monobutyl ester (TE₂B₁) and/or citric acid-monoglycidyl ether-dibutyl ester (TE₁B₂). TE₂B₁ or TE₁B₂ is obtained by ring-opening reaction of citric acid-monobutyl ester (or citric acid-dibutyl ester) with polyethylene glycol diglycidyl ether.

### CROSS-REFERENCE TO RELATED APPLICATIONS

This application claims priority to Chinese Patent Application No. 202311736271.9, filed on Dec. 18, 2023, the contents of which are hereby incorporated by reference.

### TECHNICAL FIELD

The present disclosure relates to the technical field of plastic additives, and in particular to a citric acid-glycidyl ether-butyl ester plasticizer and a preparation method thereof.

### BACKGROUND

Polylactic acid (PLA) is a biodegradable and environmental-friendly polymer derived from natural resources such as corn starch, tapioca root flakes, starch or sugarcane; through a fermentation process, high-purity lactic acid is produced, and the lactic acid may be used to synthesize polylactic acid of the desired molecular weight through a chemical process. PLA is biocompatible and widely used in medical field, including as implant material, drug delivery system and tissue engineering scaffold. The hardness and strength of PLA are comparable to that of polystyrene, which makes it favorable for packaging applications and other industrial fields. With biodegradability and low environmental impact, PLA is emerging as a promising alternative to traditional plastics.

However, the processability and potential applications of PLA are severely limited by the brittleness of the material. PLA is a low toughness polymer with very low elongation at break (less than 10%). Also, the glass transition temperature (T_{g}) of PLA is high, ranging from 55 degrees Celsius (° C.) to 65° C., leaving it fragile at room temperature. One of the more common solutions is the addition of plasticizers, and a range of plasticizers have been proposed for PLA plasticization, including epoxidized vegetable oils, glycerol esters, citrate esters, isosorbide esters, and bio-based polyesters.

Citric acid is widely found in the fruits of plants such as lemons, citrus fruits and pineapples, and in the bones, muscles and blood of animals. Citrate ester plasticizers are based on citric acid and are important environmental-friendly plasticizers thanks due to their safety, non-toxicity and anti-precipitation properties, and are approved for use in the United States, the European Union and other developed countries for plastic products that come into close contact with the human body. Ideal plasticizers should be non-toxic and has good compatibility with PLA and high thermal stability; such requirements are met by citrate ester plasticizers, such as triethyl citrate (TEC), tributyl citrate (TBC), and acetyl tributyl citrate (ATBC), etc., but there is a limited range of citrate ester plasticizers with good plasticizing effect and good anti-migration properties.

Therefore, the development of a bio-based plasticizer (citric acid-glycidyl ether-butyl ester plasticizer) with better plasticizing effect, non-toxicity and environmental protection, and good anti-migration properties to broaden the types of citrate plasticizers used for PLA plasticizing is of great significance to the field of plastic additive technology.

### SUMMARY

Based on the above contents, the present disclosure provides a citric acid-glycidyl ether-butyl ester plasticizer and a preparation method thereof. The present disclosure takes citric acid, n-butanol and polyethylene glycol diglycidyl ether as raw materials to prepare citric acid-glycidyl ether-butyl ester with both long and short chain structures and epoxy groups, and the citric acid-glycidyl ether-butyl ester is added into PLA as a plasticizer to prepare a plasticized PLA film, so as to provide a promising strategy for solving the plasticizing performance problem of PLA.

In order to achieve the above objectives, the present disclosure provides the following technical schemes.

One technical scheme of the present disclosure provides a citric acid-glycidyl ether-butyl ester plasticizer, including citric acid-diglycidyl ether-monobutyl ester (TE₂B₁) and/or citric acid-monoglycidyl ether-dibutyl ester (TE₁B₂);
a structural formula of the citric acid-diglycidyl ether-monobutyl ester is shown as Formula I; and
a structural formula of the citric acid-monoglycidyl ether-dibutyl ester is shown as Formula II;

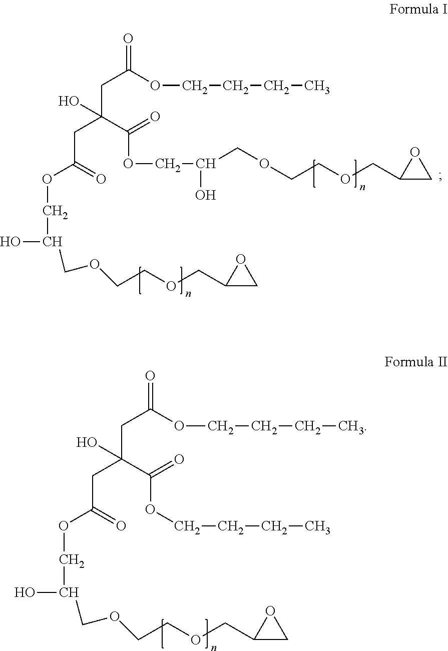

Another technical scheme of the present disclosure provides a preparation method of the citric acid-glycidyl ether-butyl ester plasticizer, including following steps:
carrying out ring-opening reaction on citric acid-monobutyl ester and polyethylene glycol diglycidyl ether to obtain the citric acid-diglycidyl ether-monobutyl ester; and
a preparation method of the citric acid-monoglycidyl ether-dibutyl ester includes following steps:
carrying out ring-opening reaction on citric acid-dibutyl ester and polyethylene glycol diglycidyl ether to obtain the citric acid-monoglycidyl ether-dibutyl ester.

Another technical scheme of the present disclosure is an application of the citric acid-glycidyl ether-butyl ester plasticizer in preparing PLA products.

Another technical scheme of the present disclosure provides a plasticized PLA material, with raw materials including the citric acid-glycidyl ether-butyl ester plasticizer.

Another technical scheme of the present disclosure provides an application of the plasticized PLA material in preparing implant materials, drug delivery systems and tissue engineering scaffolds.

The present disclosure achieves the following technical effects.

The present disclosure provides a novel bio-based citric acid-glycidyl ether-butyl ester plasticizer, which is capable of being used for plasticizing PLA.

The citric acid-glycidyl ether-butyl ester plasticizer prepared by the present disclosure is added into PLA to prepare plasticized PLA films with different formulations, and the properties of these samples are tested, with tributyl citrate used as a control. Upon addition of the plasticizers synthesized in the present disclosure, the PLA films exhibit good compatibility and plasticizing properties, as evidenced by the results of tensile and migration tests as well as T_{g} values. Among them, the PLA plasticized with 15 parts of citric acid-diglycidyl ether-monobutyl ester reaches an elongation at break of 304.6%, and the T_{g} with 20 parts of citric acid-monoglycidyl ether-dibutyl ester decreases to 39.3° C. In addition, the migration properties of the plasticizer are improved by introducing epoxy groups and increasing the molecular weight while maintaining its plasticizing effect as much as possible. Moreover, the PLA film plasticized with the plasticizer of the present disclosure has better transparency and optical properties.

### BRIEF DESCRIPTION OF THE DRAWINGS

In order to explain the embodiments of the present disclosure or the technical scheme in the prior art more clearly, the drawings needed in the embodiments will be briefly introduced below. Obviously, the drawings described below are only some embodiments of the present disclosure, and other drawings can be obtained according to these drawings without creative work for ordinary people in the field.

FIG. 1A shows a proton nuclear magnetic resonance (¹H NMR) diagram of the citric acid-glycidyl ether-butyl ester plasticizer TE₂B₁ prepared in Embodiment 1.

FIG. 1B shows a proton nuclear magnetic resonance (¹H NMR) diagram of the citric acid-glycidyl ether-butyl ester plasticizer TE₁B₂ prepared in Embodiment 2.

FIG. 2 is a Fourier Transform infrared spectroscopy (FTIR) diagram of citric acid-glycidyl ether-butyl ester plasticizers TE₂B₁ and TE₁B₂ prepared in Embodiment 1 and Embodiment 2, as well as the used raw materials of citric acid, citric acid-monobutyl ester (TB₁) and citric acid-dibutyl ester (TB₂).

FIG. 3A shows E′ curves of plasticized PLA films with different formulations in Application embodiment 1 measured by Dynamic Mechanical Analysis (DMA).

FIG. 3B is also a graph showing E′ curves of plasticized PLA films with different formulations in Application embodiment 1 measured by DMA.

FIG. 3C shows tan δ curves of plasticized PLA films with different formulations in Application embodiment 1 measured by DMA.

FIG. 3D is also a graph showing tan δ curves of plasticized PLA films with different formulations in Application embodiment 1 measured by DMA.

FIG. 4A shows the tensile test results of elongation at break of plasticized PLA films with different formulations in Application embodiment 1.

FIG. 4B shows the tensile test results of tensile strength of plasticized PLA films with different formulations in Application embodiment 1.

FIG. 5A shows the results of leaching experiments of plasticized PLA films in petroleum ether at room temperature for 24 hours (h).

FIG. 5B shows the results of leaching experiments in ethanol of plasticized PLA films at room temperature for 24 h.

FIG. 5C shows the results of leaching experiments in water of plasticized PLA films at room temperature for 24 h.

FIG. 5D shows s the results of volatility test of plasticized PLA films in an oven at 80° C. for 72 h.

FIG. 6A shows the ultraviolet-visible spectra of plasticized PLA films with different contents of TE₂B₁.

FIG. 6B shows the ultraviolet-visible spectra of plasticized PLA films with different contents of TE₁B₂ and TBC.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this present disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the present disclosure. The description and embodiments of the present disclosure are exemplary only.

The terms “including”, “comprising”, “having” and “containing” used in this specification are all open terms, which means including but not limited to.

Unless otherwise specified, “parts” in the present disclosure all mean parts by mass.

An aspect of the present disclosure provides a citric acid-glycidyl ether-butyl ester plasticizer, including citric acid-diglycidyl ether-monobutyl ester and/or citric acid-monoglycidyl ether-dibutyl ester;
a structural formula of citric acid-diglycidyl ether-monobutyl ester is shown as Formula I; and
a structural formula of the citric acid-monoglycidyl ether-dibutyl ester is shown as Formula II;

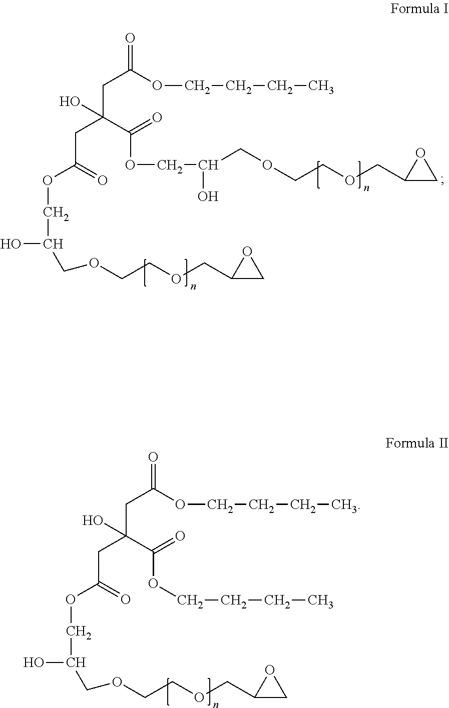

Another aspect of the present disclosure provides a preparation method of the citric acid-glycidyl ether-butyl ester plasticizer, where a preparation method of the citric acid-diglycidyl ether-monobutyl ester includes the following steps:
carrying out ring-opening reaction on citric acid-monobutyl ester and polyethylene glycol diglycidyl ether to obtain the citric acid-diglycidyl ether-monobutyl ester; and
a preparation method of the citric acid-monoglycidyl ether-dibutyl ester includes following steps:
carrying out ring-opening reaction on citric acid-dibutyl ester and polyethylene glycol diglycidyl ether to obtain the citric acid-monoglycidyl ether-dibutyl ester.

A structural formula of the citric acid-monobutyl ester is as follows:

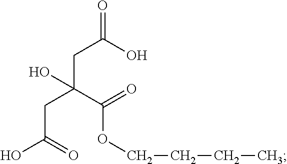 [SMILES: CCCCOC(=O)C(O)(CC(=O)O)CC(=O)O]

a structural formula of the citric acid-dibutyl ester is as follows:

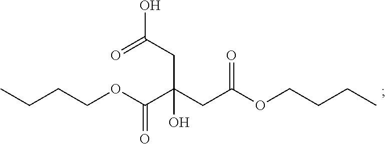 [SMILES: CCCCOC(=O)CC(O)(CC(=O)O)C(=O)OCCCC]

a structural formula of the polyethylene glycol diglycidyl ether is

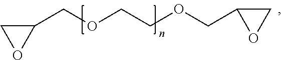

with a number average molecular weight of 300-800.

Polyethylene glycol diglycidyl ether with too high a molecular weight has a poor plasticizing effect, and too low a molecular weight tends to migrate, therefore the number average molecular weight of polyethylene glycol diglycidyl ether is preferably limited in the present disclosure to be in the range of 300-800.

In a preferred embodiment of the present disclosure, a temperature of the ring-opening reaction is 110-140° C. and a duration is 0.5-1 h.

Exceeding the above temperature or reaction duration will result in a cross-linking reaction, and falling below the specified temperature or reaction duration will result in an incomplete ring-opening reaction. Therefore, the temperature and duration of the ring-opening reaction are preferably limited to 110-140° C. and 0.5-1 h in the present disclosure.

In a preferred embodiment of the present disclosure, in a preparation process of citric acid-diglycidyl ether-monobutyl ester, a molar ratio of the citric acid-monobutyl ester to polyethylene glycol diglycidyl ether is 1:2; and in a preparation process of citric acid-monoglycidyl ether-dibutyl ester, a molar ratio of the citric acid-dibutyl ester to polyethylene glycol diglycidyl ether is 1:1.

In a further preferred embodiment of the present disclosure, the citric acid-monobutyl ester (or citric acid-dibutyl ester) and polyethylene glycol diglycidyl ether are added according to the molar ratio of citric acid-monobutyl ester to polyethylene glycol diglycidyl ether of 1:2 or the molar ratio of citric acid-dibutyl ester to polyethylene glycol diglycidyl ether of 1:1, followed by uniformly stirring, and heating for ring-opening reaction to obtain the citric acid-diglycidyl ether-monobutyl ester or the citric acid-monoglycidyl ether-dibutyl ester.

In a preferred embodiment of the present disclosure, a preparation method of the citric acid-monobutyl ester or the citric acid-dibutyl ester includes the following steps:

carrying out esterification reaction on citric acid and n-butanol to obtain the citric acid-monobutyl ester or the citric acid-dibutyl ester.

In a preferred embodiment of the present disclosure, when a product is citric acid-monobutyl ester, a molar ratio of citric acid to n-butanol is 1:1;
when a product is citric acid-dibutyl ester, a molar ratio of citric acid to n-butanol is 1:2; and
a temperature of the esterification reaction is 105-130° C., and a duration is 4-6 h.

Above or below the above temperature range of the esterification reaction will result in failure of the esterification reaction, and for a duration shorter than the above esterification reaction will result in an incomplete esterification reaction and a low yield of the product. Therefore, the present disclosure preferably limits the temperature of the esterification reaction to 105-130° C. and the duration to 4-6 h.

In a further preferred embodiment of the present disclosure, the preparation method of the citric acid-monobutyl ester or the citric acid-dibutyl ester includes the following steps:

melting citric acid, adding n-butanol according to the molar ratio of citric acid to n-butanol 1:1 or 1:2, then adding esterification catalyst, stirring well, and heating under protective atmosphere for esterification reaction to obtain the citric acid-monobutyl ester or citric acid-dibutyl ester.

A structural formula of the citric acid is

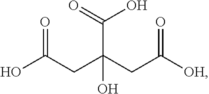 [SMILES: O=C(O)CC(O)(CC(=O)O)C(=O)O]

and a structural formula of the n-butanol is

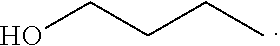

Optionally, the esterification catalyst is 1-propyl sulfonic acid-3-methylimidazole bisulfate; and the protective atmosphere is a nitrogen environment.

Optionally, the citric acid is melted, followed by adding n-butanol and esterification catalyst, and toluene is added as water-carrying agent.

Another aspect of the present disclosure provides an application of the citric acid-glycidyl ether-butyl ester plasticizer in preparing PLA products.

Another aspect of the present disclosure provides a plasticized PLA material, with raw materials including the above-mentioned citric acid-glycidyl ether-butyl ester plasticizer.

In a preferred embodiment of the present disclosure, the raw materials include, by mass, 100 parts of PLA and 5-20 parts of the citric acid-glycidyl ether-butyl ester plasticizer.

The plasticized PLA material may be a plasticized PLA film.

A preparation method of the plasticized PLA film includes the following steps: uniformly mixing PLA and the citric acid-glycidyl ether-butyl ester plasticizer to obtain a mixed material, and compressing the mixed material into a film to obtain the plasticized PLA film.

Optionally, a step of uniformly mixing PLA and the citric acid-glycidyl ether-butyl ester plasticizer to obtain the mixed material includes: stirring PLA and citric acid-glycidyl ether-butyl ester plasticizer at a temperature of 170-190° C. and a rotation speed of 60-80 revolutions per minute (rpm) for 5-7 min to obtain the mixed material.

Optionally, a temperature of the compressing is 170-190° C. and a pressure is 80-100 mega pascal (MPa).

Optionally, a thickness of the plasticized PLA film is 0.1-0.5 millimeter (mm).

Another aspect of the present disclosure provides an application of the plasticized PLA material in preparing implant materials, drug delivery systems and tissue engineering scaffolds.

Unless otherwise specified, the raw materials used in the following embodiments and application embodiments of the present disclosure may be obtained through commercial channels. Among them, PLA pellet (model 4032D) is purchased from Nature Works (melt flow rate (MFR)=7 g/10 min, 190° C., 2.16 kg, Minnetonka); citric acid is purchased from Beijing Mreda Technology Co., Ltd.; n-butanol (99.5%) is purchased from Beijing Innochem Technology Co., Ltd.; the 1-propyl sulfonic acid-3-methylimidazole bisulfate ionic liquid (99%) is purchased from Lanzhou Institute of Chemical Physics, Chinese Academy of Sciences. Tributyl citrate (TBC) and polyethylene glycol diglycidyl ether (PEGDE, with a number average molecular weight of about 500) are purchased from Shanghai Aladdin Bio-Chem Technology Co., LTD; and toluene is purchased from Beijing Institute of Chemical Reagents Co., Ltd.

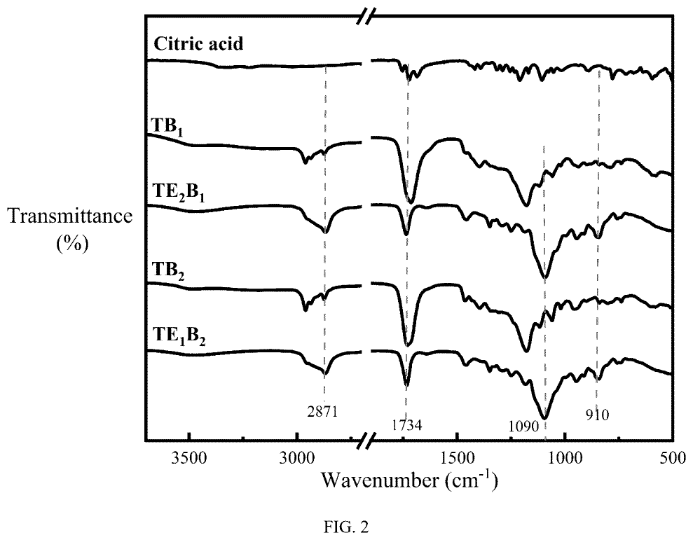

The citric acid-monobutyl ester used in the following embodiments of the present disclosure is prepared by the following steps: citric acid (1 mol, 1 equivalent) and n-butanol (1 mol, 1 equivalent) are added into a 500 milliliters (mL) three-necked flask with a magnetic stirrer; the flask is connected with a condenser tube and introduced with condensed water. Then, the esterification catalyst 1-propylsulfonic acid-3-methylimidazole hydrogen sulfate (0.1 mol, 0.1 equivalent) and 60 ml of toluene with aqueous agent are added. With continuous stirring at 120° C. in a nitrogen atmosphere, the water generated in the reaction is continuously carried by the toluene via the condenser tube into a collection device, and the toluene is simultaneously returned to the three-necked flask. After 6 h of reaction, no more water is generated, indicating that the esterification reaction is completed. The product is cooled and separated by a partition funnel to take the upper layer, and then the remaining water and toluene are removed by vacuum evaporation to obtain the product citric acid-monobutyl ester (TB₁, the infrared spectra are shown in FIG. 2).

The preparation method of the citric acid-dibutyl ester (TB₂, whose infrared spectrum is shown in FIG. 2) used in the embodiments of the present disclosure is different from the preparation method of citric acid-monobutyl ester mentioned above only in that the consumption of n-butanol is 2 mol and 2 equivalents.

The present disclosure will be further illustrated by embodiments.

### Embodiment 1

### Preparation of citric acid-glycidyl ether-butyl ester plasticizer TE₂B₁ (citric acid-diglycidyl ether-monobutyl ester)

Citric acid-monobutyl ester (1 mol, 1 equivalent) and polyethylene glycol diglycidyl ether (2 mol, 2 equivalent) are placed in a 500 mL three-neck flask with a magnetic stirrer and stirred continuously at 120° C. for 1 h to obtain TE₂B₁.

### Embodiment 2

### Preparation of citric acid-glycidyl ether-butyl ester plasticizer TE₁B₂ (citric acid-monoglycidyl ether-dibutyl ester)

Citric acid-dibutyl ester (1 mol, 1 equivalent) and polyethylene glycol diglycidyl ether (1 mol, 1 equivalent) are placed in a 500 mL three-neck flask with a magnetic stirrer and stirred continuously at 120° C. for 1 h to obtain TE₁B₂.

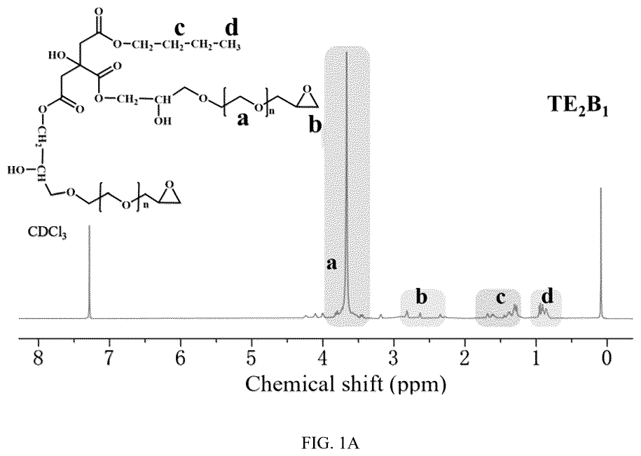

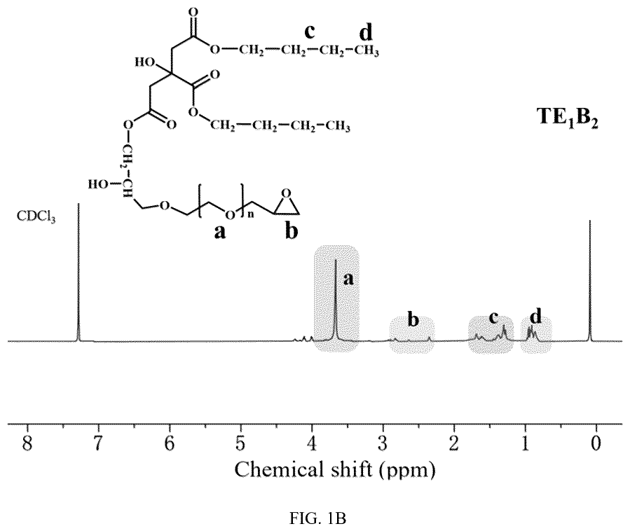

FIG. 1A and FIG. 1B are the ¹H NMR diagrams of citric acid-glycidyl ether-butyl ester plasticizers prepared in Embodiment 1 and Embodiment 2. As shown in FIG. 1A and FIG. 1B, there are 2.0-3.0 parts per million (ppm) epoxy group peaks and 3.5-3.7 ppm ether bond peaks in the spectrograms of TE₂B₁ and TE₁B₂, and n-butyl peaks of 1.2-1.5 ppm and 0.6-1.0 ppm may be observed in the spectrograms of TE₂B₁ and TE₁B₂, and the results prove the successful synthesis of citric acid-glycidyl ether-butyl ester plasticizer.

FIG. 2 is a Fourier Transform infrared spectroscopy (FTIR) diagram of citric acid-glycidyl ether-butyl ester plasticizer prepared in Embodiment 1 and Embodiment 2, as well as raw materials of citric acid, citric acid-monobutyl ester and citric acid-dibutyl ester. As observed from FIG. 2, ester group peaks of citric acid-monobutyl ester and citric acid-dibutyl ester may be observed at 1734 cm⁻¹, and the appearance of the peaks indicates the successful esterification reaction. The C—O—C— stretching vibration peak of epoxy group at 910 cm⁻¹ and the absorption peak of ether bond at 1090 cm⁻¹ appear in TE₂B₁ and TE₁B₂, and the infrared absorption peak of ester group is shifted from the first step, indicating the successful synthesis of plasticizer.

A comparison of the color of plasticizers TE₂B₁ and TE₁B₂, derived from citrate-glycidyl ester-butyl ester in Embodiments 1 and 2, with commercial TBC plasticizers reveals that both TE₂B₁ and TE₁B₂ are transparent liquids at room temperature, albeit slightly darker than TBC.

### Application Embodiment 1

### Preparation of Plasticized PLA Film

Formulation: using 100 parts of PLA as the base material and the citric acid-glycidyl ether-butyl ester plasticizers TE₂B₁ and TE₁B₂ prepared in Embodiment 1 as plasticizers (the dosage of plasticizer is 5-20 parts), plasticized PLA films with different formulations are obtained as the experimental group. The ratio statistics of the formulations are shown in Table 1.

In addition, using 100 parts of PLA as base material and TBC as plasticizer (the amount of plasticizer is 5-20 parts), plasticized PLA films with different formulations are obtained as control group. The ratio statistics of the formulations are also shown in Table 1.

Preparation method: firstly, an internal mixer (Haake Rheometer) is used to stir for 5-7 min at the temperature of 175° C. and the rotation speed of 60 rpm, then the obtained homogeneous mixture is compressed into a film with a thickness of 0.5 mm by a hydraulic press. The compression parameters are 180° C. and 80 MPa.

TABLE 1
Formulation table of plasticized PLA films

| | Content of components | | | |
|---|---|---|---|---|
| Names of plasticized | PLA | TE₂B₁ | TE₁B₂ | TBC |
| PLA films | (parts) | (parts) | (parts) | (parts) |
| PLA/5TE₂B₁ | 100 | 5 | — | — |
| PLA/10TE₂B₁ | 100 | 10 | — | — |
| PLA/15TE₂B₁ | 100 | 15 | — | — |
| PLA/20TE₂B₁ | 100 | 20 | — | — |
| PLA/5TE1B₂ | 100 | — | 5 | — |
| PLA/10TE₁B₂ | 100 | — | 10 | — |
| PLA/15TE₁B₂ | 100 | — | 15 | — |
| PLA/20TE₁B₂ | 100 | — | 20 | — |
| PLA/5TBC | 100 | — | — | 5 |
| PLA/10TBC | 100 | — | — | 10 |
| PLA/15TBC | 100 | — | — | 15 |
| PLA/20TBC | 100 | — | — | 20 |

Effect verification embodiment (performance test of plasticized PLA film (sample) prepared in Application embodiment 1)

### 1. Plasticizing Performance Test

Plasticizing performance is determined by evaluating the decrease of polymer T_{g}. The T_{g} value of plasticized PLA film is determined by the peak value of tan δ of its dynamic mechanical analysis (DMA) curve.

Test method: the DMA is carried out on DMA7100 (Hitachi, Japan) to evaluate the glass transition temperature T_{g} of the plasticized PLA film (40 mm×10 mm×0.5 mm). The DMA analysis is carried out between −30° C. and 120° C., and the heating rate is 5° C./min.

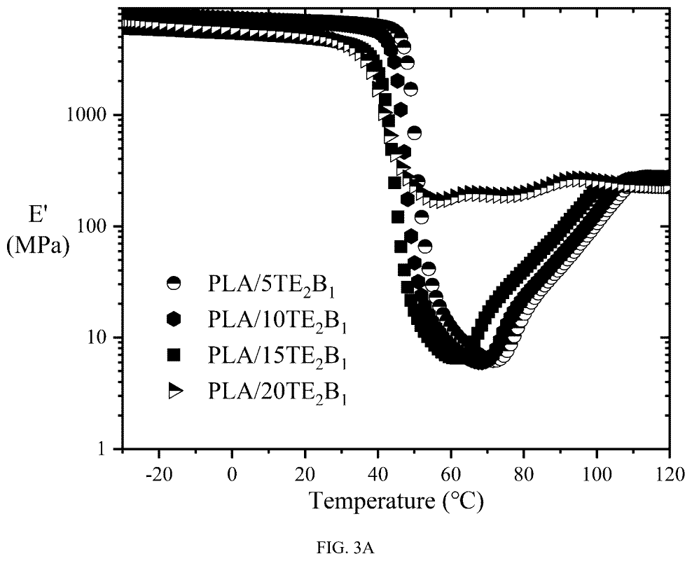

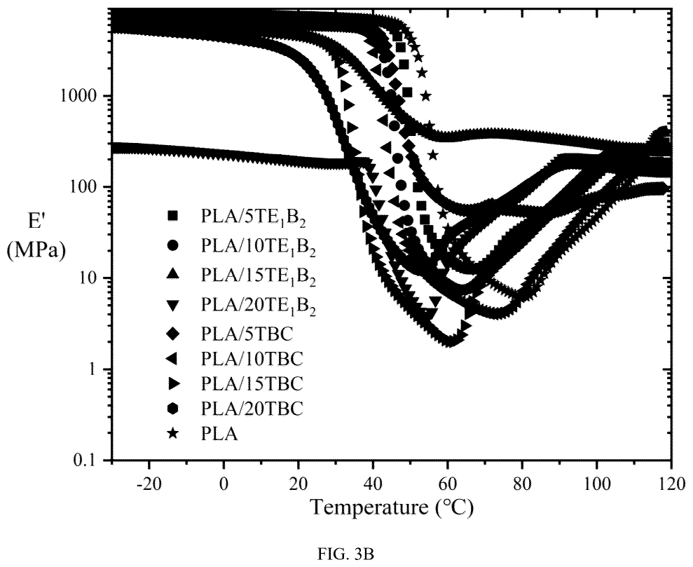

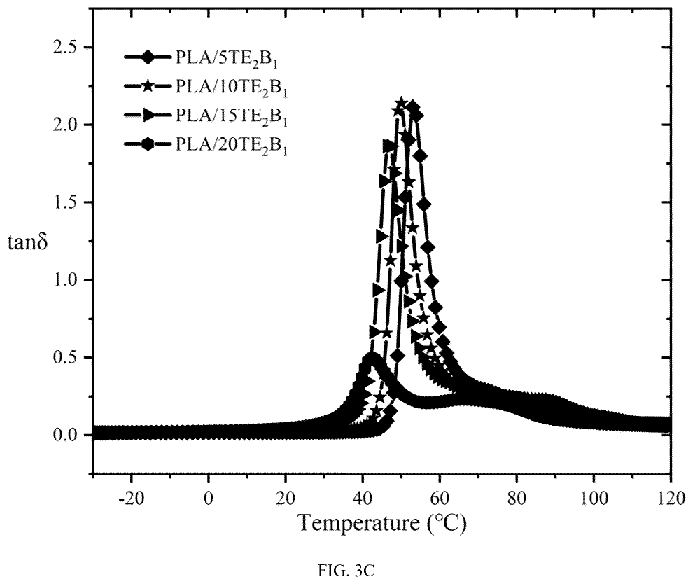

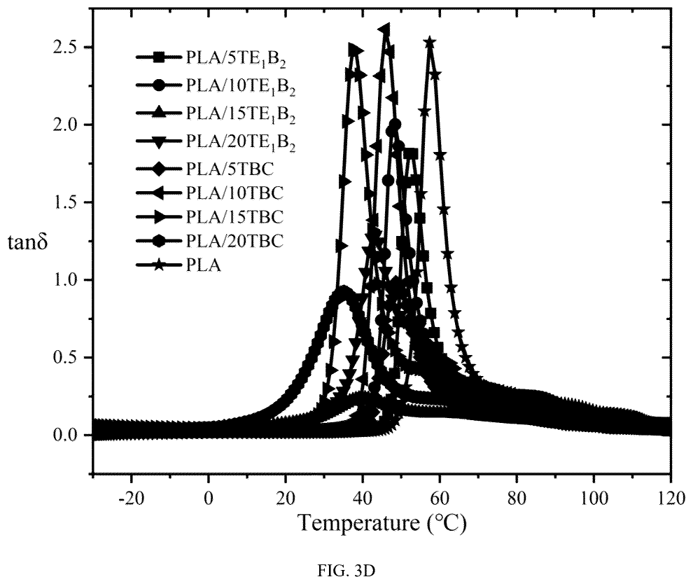

Test results: the DMA curves of plasticized PLA films with different formulations in the present disclosure as shown in FIG. 3A, FIG. 3B, FIG. 3C and FIG. 3D, and FIG. 3A to FIG. 3D suggest that the storage modulus of the samples remains stable in the temperature range of −30° C. to 40° C., with no obvious decrease, so this is the temperature range where PLA and plasticized PLA materials remain thermally and mechanically stable. As for E′, there is a sharp drop of 2-3 orders of magnitude occurred between 40° C. and 70° C., which is due to the glass transition region of PLA. The second turning point occurs between 70° C. and 90° C., and the storage modulus begins to rise due to the cold crystallization process. In this stage, the molecules will be rearranged, leading to a greater degree of stacking, which will lead to a slight increase in thermo-mechanical properties.

The T_{g} of unplasticized PLA samples is about 57.39° C., and the tan δ peak is very narrow, with the maximum value of about 2.53, while plasticized samples show a wider peak, and the tan δ value is obviously reduced. The T_{g} value of 20 phr TE₂B₁ and TE₁B₂ plasticized PLA films measured by DMA is 42.25° C. and 43.27° C., respectively, which is 15.14° C. and 14.12° C. lower than that of pure PLA. The glass transition temperature of PLA/15TE₁B₂ is the lowest (39.81° C.), reduced by 17.58° C.

### 2. Mechanical Properties Test

Test method: China standard GB/T1040.1-2006 is used to evaluate the mechanical properties of plasticized PLA films, and the elongation at break (%) and tensile strength (MPa) are measured. The measurement is carried out on an electronic universal testing machine controlled by a microcomputer (CMT6104, China). The jig spacing is 30 mm and the drawing speed is 20 mm/min. Each group of samples is tested for 10 times. The average is taken and the error is calculated.

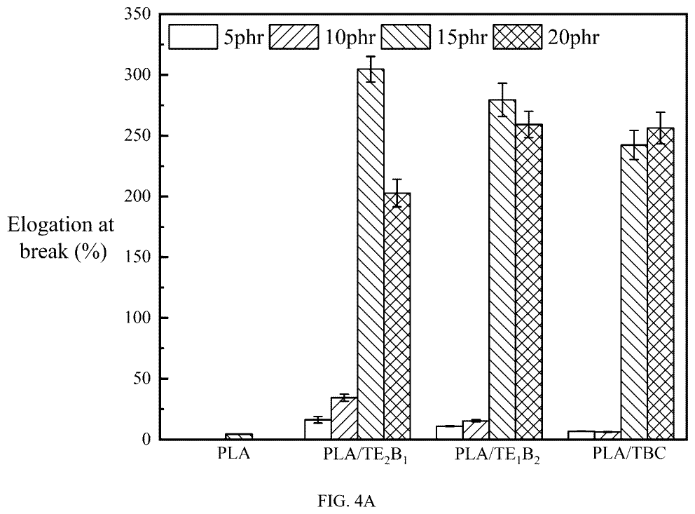

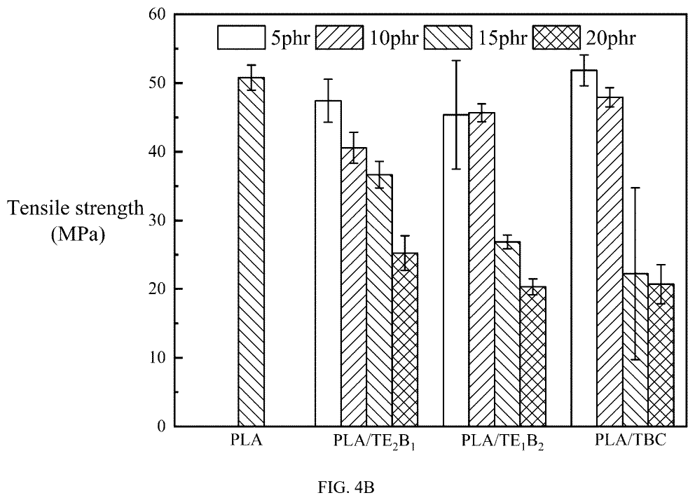

Test results: the tensile test results of plasticized PLA films with different formulations in Application embodiment 1 of the present disclosure are shown in FIG. 4A, FIG. 4B, and Table 2. The decrease of tensile strength is observed after adding plasticizer, which is consistent with the general expectation of plasticized polymers. Among the PLA films with 15 phr plasticizer, the PLA film with 15 phr TE₂B₁ has the highest tensile strength (36.69 MPa), and the largest decrease in tensile strength (20.32 MPa) is observed when 20 phr TE₁B₂ is added.

Elongation at break is a necessary parameter to evaluate the relationship between plasticizing efficiency and plasticizer function. By comparing the elongation at break of several plasticized films with different formulations, it is found that the sample with 15 phr of TE₂B₁ has the best effect (304.64%), followed by PLA/15TE₁B₂ (279.48%), PLA/20TE₁B₂ (259.24%) and PLA/20TE₂B₁ (202.84%).

Among the plasticizers tested, PLA/15TE₂B₁ has the best plasticizing efficiency. And with the occurrence of yield, the yield point appears and the stress turns white. With the content of plasticizer increasing to 20 phr, the elongation at break decreases, which may be due to the phase separation caused by the incompatibility between plasticizer and PLA.

TABLE 2
Tensile strength and elongation at break of plasticized
PLA films with different formulations

| | Elongation at break (%) | | | |
|---|---|---|---|---|
| Pure PLA | 4.57 ± 0.14 | | | |
| Addition amount (phr) | 5 | 10 | 15 | 20 |
| TE₂B₁ | 16.29 ± 2.69 | 34.45 ± 2.91 | 304.64 ± 10.52 | 202.84 ± 11.33 |
| TE₁B₂ | 11.02 ± 0.35 | 15.49 ± 0.91 | 279.48 ± 13.61 | 259.24 ± 10.78 |
| TBC | 6.87 ± 0.1 | 6.33 ± 0.31 | 242.34 ± 11.91 | 256.33 ± 12.95 |

| | Tensile strength (MPa) | | | |
|---|---|---|---|---|
| Pure PLA | 50.80 ± 1.82 | | | |
| Addition amount (phr) | 5 | 10 | 15 | 20 |
| TE₂B₁ | 47.42 ± 3.12 | 40.59 ± 2.25 | 36.69 ± 1.95 | 25.22 ± 2.52 |
| TE₁B₂ | 45.39 ± 7.88 | 45.68 ± 1.31 | 26.85 ± 0.98 | 20.32 ± 1.16 |
| TBC | 51.85 ± 2.25 | 47.95 ± 1.4 | 22.25 ± 12.51 | 20.71 ± 2.84 |

### 3. Thermal Stability Test

PLA products are required to have good thermal stability in production and practical application, and the thermogravimetric analysis (TGA) is used to evaluate the thermal stability of PLA films.

Test method: TGA is carried out on STA7200 (Hitachi, Japan) to test the thermal stability of plasticized PLA films. In each test, the weight loss of the sample is recorded in nitrogen atmosphere. The measuring temperature range is 40-700° C., and the heating rate is 10° C./min.

Test results: the TGA analysis results of plasticized PLA films with different formulations in Application embodiment 1 of the present disclosure are shown in Table 3. The T_{d-5%} of unplasticized PLA is 343.98° C. and T_{d-max} is 376.58° C. However, adding a certain amount of plasticizer to PLA will lead to the decrease of T_{d-5%} and T_{d-max} value, the greater the amount added, the lower these values, because the plasticizer itself will decompose at a lower temperature. For example, the measured T_{d-5%} of samples with 15 phr of TE₂B₁ and TE₁B₂ are 295.78° C. and 293.93° C., which are 48.2° C. and 50.05° C. lower than those of unplasticized PLA, respectively. The thermal degradation results of plasticized PLA films with different formulations, including T_{d-5%}, T_{d-50%} and T_{d-max} values, are summarized in Table 3.

TABLE 3
Thermal degradation temperature of PLA
films with different formulations

| | T_{d-5%} | T_{d-50%} | T_{d-max} |
|---|---|---|---|
| Test samples | (° C.) | (° C.) | (° C.) |
| PLA/5TE₂B₁ | 305.71 | 368.17 | 377.61 |
| PLA/10TE₂B₁ | 297.98 | 362.16 | 377.89 |
| PLA/15TE₂B₁ | 295.78 | 359.05 | 376.80 |
| PLA/20TE₂B₁ | 300.79 | 363.36 | 376.27 |
| PLA/5TE₁B₂ | 326.18 | 372.45 | 377.31 |
| PLA/10TE₁B₂ | 300.27 | 357.32 | 366.05 |
| PLA/15TE₁B₂ | 293.93 | 344.60 | 331.53 |
| PLA/20TE₁B₂ | 295.13 | 344.98 | 345.56 |
| PLA/5TBC | 332.90 | 374.11 | 378.96 |
| PLA/10TBC | 299.27 | 371.74 | 377.73 |
| PLA/15TBC | 270.77 | 364.74 | 368.24 |
| PLA/20TBC | 250.27 | 368.45 | 375.21 |

T_{d-5%} and T_{d-50%} respectively represent the temperature at which 5% and 50% weight loss occurs, and T_{d-max} represents the maximum weightlessness temperature (the peak temperature of derivative thermogravimetry (DTG)).

### 4. Migration Performance Test

In addition to plasticizing efficiency, plasticizers should also have migration resistance. Plasticized materials with high plasticizer content may migrate, resulting in sticky surface, thus affecting the practical application of PLA products.

Test method: volatility test and leaching experiment are carried out according to ASTMD1239-98 and ISO176:2005 standards. The volatility experiment is carried out at 80° C. for 72 h and the leaching experiment is carried out in water, anhydrous ethanol and petroleum ether, respectively.

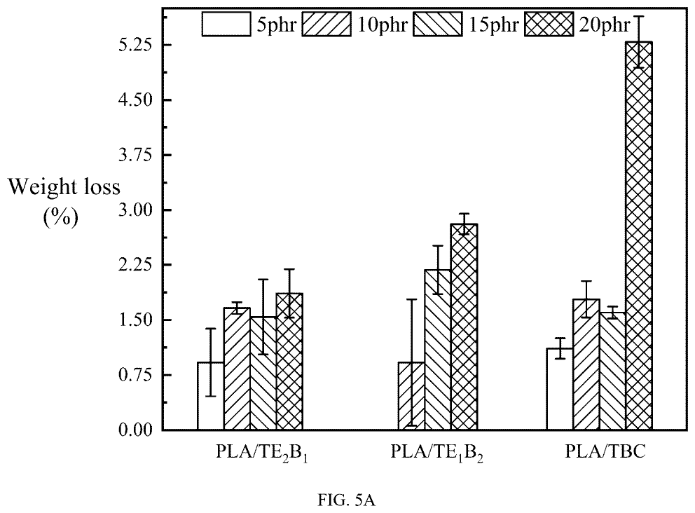

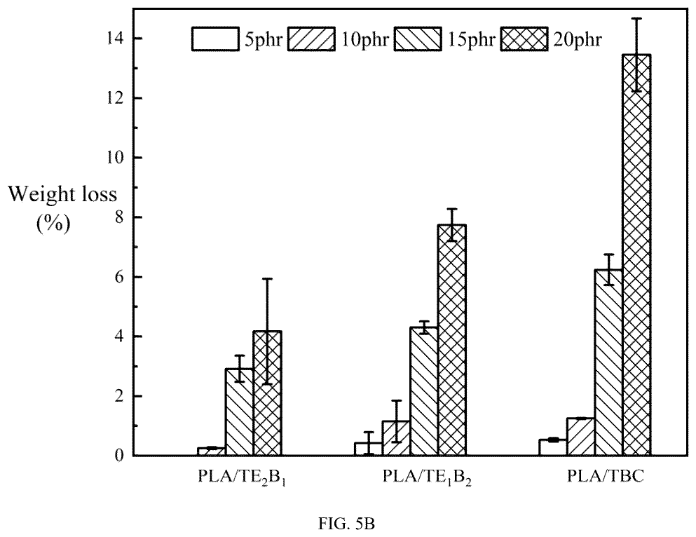

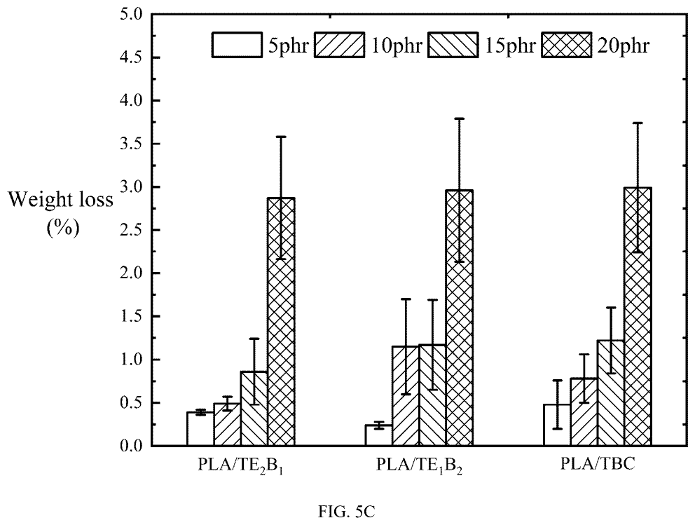

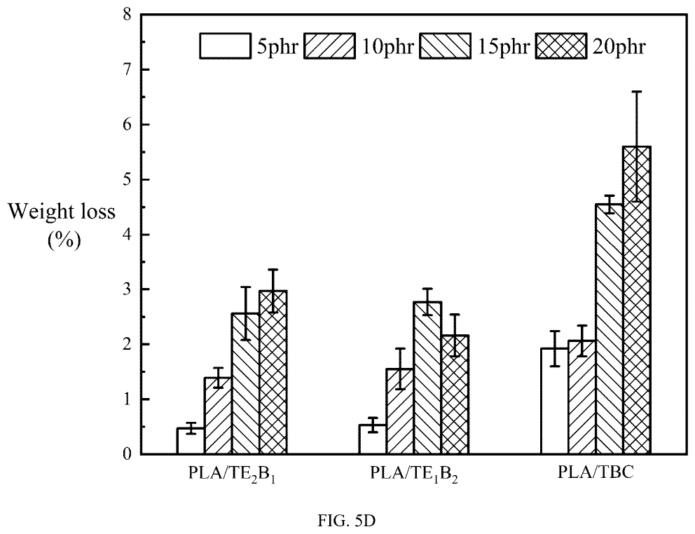

Test results: the volatility test and leaching experimental results of plasticized PLA membranes with different formulations in Application embodiment 1 of the present disclosure are shown in FIG. 5A-FIG. 5D, in which FIG. 5A, FIG. 5B and FIG. 5C are the leaching experimental results of soaking in petroleum ether, ethanol and water for 24 h at room temperature, and FIG. 5D shows the volatility experimental results of standing in an oven at 80° C. for 72 h. FIG. 5A-FIG. 5C show that when the amount of plasticizer is increased from 5 phr to 20 phr, the mass loss also shows an increasing trend, which accords with the general law that the anti-migration ability of the sample gradually decreases with the increase of the amount of plasticizer. For example, FIG. 5C shows that the mass loss increases from 0 to 4.17% when the amount of PLA/TE₂B₁ is increased from 5 phr to 20 phr. Moreover, PLA plasticized by plasticizer with high molecular weight has better migration resistance, and PLA plasticized by TE₁B₂ with small molecular weight of 5, 10, 15 and 20 phr has higher mass loss (0.42%, 1.15%, 4.3% and 7.74%, respectively). In addition, the epoxy group introduced into the plasticizer may improve the intermolecular force, thus improving the migration resistance of the plasticizer. The experimental results also conform to the changing trend of the number of epoxy groups in the plasticizer structure. FIG. 5D shows that only a few molecules volatilize after being placed in the oven for 72 h due to the stronger interaction between TE₂B₁ and TE₁B₂ and PLA, showing a low volatilization value.

### 5. Optical Performance Test

PLA film used in packaging field require good optical properties. Two important optical performance indexes are transmittance and haze. The light transmittance may be qualitatively analyzed by visual inspection, and may also be characterized by haze meter and UV-Vis spectrum.

Testing method: the optical properties of plasticized PLA film are tested by using UV-Vis near infrared spectrophotometer (UV-3600, Japan), and the transmittance curve in the range of 200-800 nm is obtained. In addition, the haze and light transmittance of PLA film are measured by WGT-S haze meter.

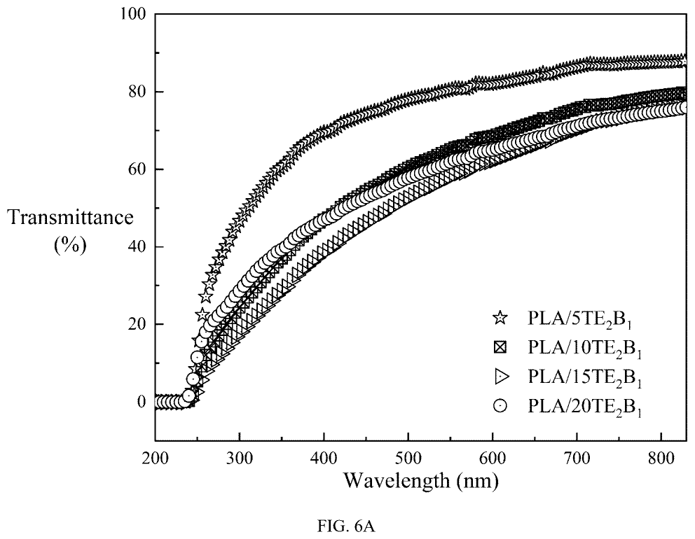

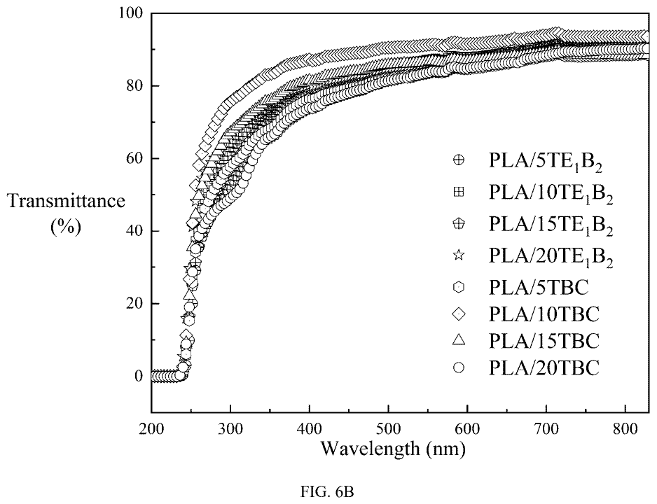

Test results: the UV-visible spectra of plasticized PLA films with different formulations in Application embodiment 1 of the present disclosure are shown in FIG. 6A and FIG. 6B, which show that the transmittance of samples gradually increases in the order of TE₂B₁ and TE₁B₂, among which TE₁B₂ has the highest transmittance, and the transmittance of each formulation exceeds 85% in the range of 600-800 nm.

Moreover, the haze values and light transmittance of PLA films with different formulations are shown in Table 4. From Table 4, it is observed that the light transmittance of all samples with plasticizer formulations is above 90%, and the light transmittance of PLA/15TE₂B₁ is the highest, reaching 93.9%.

TABLE 4
Comparison of haze and transmittance of PLA samples

| | | | | |
|---|---|---|---|---|
| Addition amount | 5 | 10 | 15 | 20 |

Haze values (%)

| | | | | |
|---|---|---|---|---|
| PLA/TE₂B₁ | 7.2 ± 0.9 | 13.9 ± 2.5 | 19.6 ± 2.1 | 28.1 ± 0.9 |
| PLA/TE₁B₂ | 6.3 ± 1.2 | 6.6 ± 0.7 | 5.8 ± 0.4 | 16.6 ± 0.9 |
| PLA/TBC | 5.8 ± 1.8 | 3.5 ± 0.8 | 4.6 ± 0.4 | 4.0 ± 0.3 |

Transmittance (%)

| | | | | |
|---|---|---|---|---|
| PLA/TE₂B₁ | 93.3 ± 0.2 | 93.7 ± 0.1 | 93.9 ± 0.2 | 93.1 ± 0.4 |
| PLA/TE₁B₂ | 92.6 ± 0.4 | 93.1 ± 0.2 | 93.2 ± 0.1 | 92.2 ± 0.1 |
| PLA/TBC | 91.5 ± 0.8 | 92.7 ± 0.3 | 90.6 ± 0.4 | 92.9 ± 0.2 |

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the present disclosure.

What is claimed is:

1. A citric acid-glycidyl ether-butyl ester plasticizer, comprising citric acid-diglycidyl ether-monobutyl ester and/or citric acid-monoglycidyl ether-dibutyl ester;
a structural formula of the citric acid-diglycidyl ether-monobutyl ester is shown as Formula I;
a structural formula of the citric acid-monoglycidyl ether-dibutyl ester is shown as Formula II;

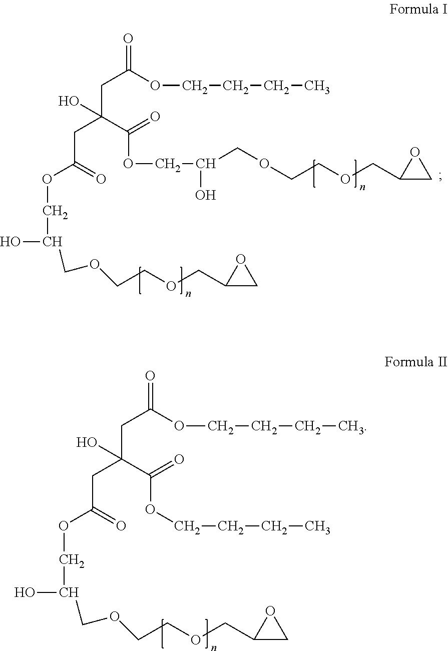

2. A preparation method of the citric acid-glycidyl ether-butyl ester plasticizer according to claim 1, wherein a preparation method of the citric acid-diglycidyl ether-monobutyl ester comprises following steps:
carrying out ring-opening reaction on citric acid-monobutyl ester and polyethylene glycol diglycidyl ether to obtain the citric acid-diglycidyl ether-monobutyl ester; and
a preparation method of the citric acid-monoglycidyl ether-dibutyl ester comprises following steps:
carrying out ring-opening reaction on citric acid-dibutyl ester and polyethylene glycol diglycidyl ether to obtain the citric acid-monoglycidyl ether-dibutyl ester.

3. The preparation method of the citric acid-glycidyl ether-butyl ester plasticizer according to claim 2, wherein a temperature of the ring-opening reaction is 110-140° C. and a duration is 0.5-1 h.

4. The preparation method of the citric acid-glycidyl ether-butyl ester plasticizer according to claim 2, wherein in a preparation process of the citric acid-diglycidyl ether-monobutyl ester, a molar ratio of the citric acid-dibutyl ester to the polyethylene glycol diglycidyl ether is 1:2; and in a preparation process of the citric acid-monoglycidyl ether-dibutyl ester, a molar ratio of the citric acid-dibutyl ester to the polyethylene glycol diglycidyl ether is 1:1.

5. The preparation method of the citric acid-glycidyl ether-butyl ester plasticizer according to claim 2, wherein a preparation method of the citric acid-monobutyl ester or the citric acid-dibutyl ester comprises following steps:
carrying out esterification reaction on citric acid and n-butanol to obtain the citric acid-monobutyl ester or the citric acid-dibutyl ester.

6. The preparation method of the citric acid-glycidyl ether-butyl ester plasticizer according to claim 5, wherein when a product is the citric acid-monobutyl ester, a molar ratio of citric acid to n-butanol is 1:1;
when a product is the citric acid-dibutyl ester, a molar ratio of citric acid to n-butanol is 1:2; and
a temperature of the esterification reaction is 105-130° C.

7. A plasticized PLA material, with raw materials comprising the citric acid-glycidyl ether-butyl ester plasticizer according to claim 1.

8. The plasticized PLA material according to claim 7, wherein the raw materials comprise 100 parts of PLA and 5-20 parts of citric acid-glycidyl ether-butyl ester plasticizer in parts by mass.
